(19) United States
(12) Patent Application Publication
(10) Pub. No.: US 2025/0143638 A1
(43) Pub. Date: May 8, 2025

(54) METHOD AND APPARATUS FOR EVALUATING ANATOMICAL ADAPTATION OF IMPLANTABLE ORTHOPEDIC MEDICAL DEVICE

(71) Applicant: THE FOURTH MEDICAL CENTER OF PLA GENERAL HOSPITAL, Beijing (CN)

(72) Inventors: Jiantao LI, Beijing (CN); Menglin WANG, Beijing (CN); Wei ZHANG, Beijing (CN); Hao ZHANG, Beijing (CN); Wanheng LIU, Beijing (CN); Cheng XU, Beijing (CN); Kaixuan WANG, Beijing (CN); Zicheng ZHANG, Beijing (CN); Yanpeng ZHAO, Beijing (CN); Daofeng WANG, Beijing (CN); Zhengfeng JIA, Beijing (CN); Weilu GAO, Beijing (CN); Meng LI, Beijing (CN); Xiaomeng REN, Beijing (CN); Wupeng ZHANG, Beijing (CN); Licheng ZHANG, Beijing (CN); Peifu TANG, Beijing (CN)

(21) Appl. No.: 18/958,478

(22) Filed: Nov. 25, 2024

Related U.S. Application Data
(63) Continuation-in-part of application No. 18/941,212, filed on Nov. 8, 2024, now abandoned.

(30) Foreign Application Priority Data
Nov. 8, 2023 (CN) 202311476899.X

(51) Int. Cl. A61B 5/00

(52) U.S. Cl. CPC A61B 5/4851; A61B 5/7264

(57) ABSTRACT

Provided are a method and apparatus for evaluating anatomical adaptation of an implantable orthopedic medical device. The method includes: obtaining an anatomical adaptation region of a to-be-evaluated bone with the implantable orthopedic medical device; determining a baseline of the anatomical adaptation region and a baseline of the implantable orthopedic medical device for position registration, and conducting the position registration for the anatomical adaptation region and the implantable orthopedic medical device based on the baselines; and evaluating adhesion of the to-be-evaluated bone to the implantable orthopedic medical device by calculating a distance from each point on an inner curved surface of the implantable orthopedic medical device to the anatomical adaptation region. This application can be used to conduct the matching analysis and adhesion evaluation of implantable orthopedic medical devices to a bone before a surgery and guide the selection of an implantable orthopedic medical device in advance.

### CROSS-REFERENCE TO RELATED APPLICATION

This patent application is a continuation-in-part (CIP) application of U.S. Ser. No. 18/941,212 filed on Nov. 8, 2024, and that application claims the benefit and priority of Chinese Patent Application No. 202311476899.X, filed with the China National Intellectual Property Administration on Nov. 8, 2023, the disclosures of which are incorporated by reference herein in its entirety as part of the present application.

### TECHNICAL FIELD

The present disclosure belongs to the technical field of medical science, and specifically relates to a method and apparatus for evaluating anatomical adaptation of an implantable orthopedic medical device.

### BACKGROUND

Implantable orthopedic medical devices refer to a wide class of implants for human bone replacement, repair, supplementation, and filling. Implantable orthopedic medical devices are developed for the maintenance, support, and repair of human bones. Implantable orthopedic medical devices include plate-screw systems, artificial implants, prostheses, or the like. Implantable orthopedic medical devices are high-value consumables commonly used in clinical practice, and are also main technical tools for surgical therapy and major practical demands for maintaining the people's health.

Implantable orthopedic medical devices vary greatly in morphology. Morphological characteristics of an implantable orthopedic medical device need to match a structural morphology of the human body to avoid the occurrence of complications of internal fixation. Therefore, it is of great significance for the treatment of diseases to conduct the matching analysis and adhesion evaluation of implantable orthopedic medical devices to a structure of the human body before a surgery and guide the selection of an implantable orthopedic medical device in advance.

### SUMMARY

In order to solve the above-mentioned problems in the prior art, the present disclosure provides a method and apparatus for evaluating anatomical adaptation of an implantable orthopedic medical device.

To allow the above objective, the present disclosure adopts the following technical solutions:

In a first aspect, the present disclosure provides a method for evaluating anatomical adaptation of an implantable orthopedic medical device, including the following steps:
obtaining a first point cloud data of a target bone and a second target point cloud data of a implantable orthopedic medical device;
performing a first determination for determining an anatomical adaptation region of the target bone with the implantable orthopedic medical device based on the first point cloud data and the second target point cloud data;
performing a second determination for determining a baseline of the anatomical adaptation region and a baseline of the implantable orthopedic medical device for position registration; conducting the position registration based on the baseline of the anatomical adaptation region and the baseline of the implantable orthopedic medical device;
performing a first calculation for calculating a minimum distance from each point on an inner curved surface of the implantable orthopedic medical device to the anatomical adaptation region;
performing a second calculation for calculating an average value of minimum distances, and comparing the average value with a predetermined threshold;
in response to the average value being less than or equal to the predetermined threshold, repairing the target bone with the internal vegetation equipment corresponding to the second target point cloud data.

In some embodiments, the method further includes the following steps:

in response to the average value being greater than the predetermined threshold:
generating a distance-based heat map based on the minimum distances and adjusting the second target point cloud data according to the distance-based heat map;
repeating the first determination, the second determination, the position registration, the first calculation, and the second calculation on the adjusted second target cloud data until the adjusted second target cloud data is less than or equal to the predetermined threshold;
repairing the target bone with an internal vegetation equipment manufactured according to the adjusted second target point cloud data.

Further, a process for determining the anatomical adaptation region of the target bone with the implantable orthopedic medical device includes:
conducting principal component analysis for point cloud data of the bone to obtain a first principal axis, a second principal axis, and a third principal axis that are perpendicular to each other and correspond to length, width, and height directions of the bone, respectively;
selecting a point p at one end of the bone, and making a plane 1 crossing the point p with the first principal axis as a normal line;
translating the plane 1 toward the other end of the bone at a distance to obtain a plane 2;
selecting points p₁ and p₂ at left and right sides of the point P in the plane 1, respectively, making a perpendicular line crossing the point P for a straight line pip₂ with a foot point of p₀, and making a plane 3 crossing the point p₀ with the perpendicular line pp₀ as a normal line; and
translating the plane 3 backward at a distance to obtain a plane 4, and intercepting a surface of the bone with the planes 1, 2, and 4 to obtain the anatomical adaptation region.

Further, a process for determining the baseline of the anatomical adaptation region includes:
calculating a centroid of the bone;
making a straight line that crosses the centroid and is parallel to the first principal axis; and
making a plane 5 that crosses the point p and the straight line, and determining an intersecting line between the plane 5 and the anatomical adaptation region as the baseline of the anatomical adaptation region.

Further, a process for determining the baseline of the implantable orthopedic medical device includes:
obtaining point cloud data of the inner curved surface of the implantable orthopedic medical device based on point cloud data of the implantable orthopedic medical device;
conducting principal component analysis for the point cloud data of the implantable orthopedic medical device to obtain a first principal axis, a second principal axis, and a third principal axis that are perpendicular to each other and correspond to length, width, and height directions of the implantable orthopedic medical device, respectively;
calculating a centroid of the implantable orthopedic medical device, and making a plane crossing the centroid with the second principal axis of the implantable orthopedic medical device as a normal line to obtain a projection plane; and
projecting a point cloud of the inner curved surface of the implantable orthopedic medical device to the projection plane to obtain the baseline of the implantable orthopedic medical device.

Further, a process for obtaining the point cloud data of the inner curved surface of the implantable orthopedic medical device includes:
selecting any point on the inner curved surface of the implantable orthopedic medical device, and calculating a normal vector v₀ crossing the point;
calculating a normal vector v₁ of each point crossing the implantable orthopedic medical device, where i=1, 2, . . . , N, and N is a number of data points of the implantable orthopedic medical device; and
calculating an included angle between v₁ and v₀, and constituting the point cloud data of the inner curved surface of the implantable orthopedic medical device with points corresponding to all included angles smaller than a set threshold.

Further, a process for conducting the position registration based on the baseline of the anatomical adaptation region and the baseline of the implantable orthopedic medical device includes:
conducting registration for the baseline of the anatomical adaptation region and the baseline of the implantable orthopedic medical device by iterative closest point (ICP) to obtain a transformation matrix R and a translation vector V; and
subjecting a point cloud matrix of the implantable orthopedic medical device to the following transformation:

$${A}^{\sim} = A * R + V$$

where A and A⁻ represent point cloud matrices of the implantable orthopedic medical device before and after the transformation, respectively.

Further, after the position registration is conducted, the method further includes: subjecting the implantable orthopedic medical device to attitude adjustment according to the following steps:
S1, making the implantable orthopedic medical device rotate left and right;
S1.1, making a straight line that crosses a centroid of the implantable orthopedic medical device and is parallel to a first principal axis of the implantable orthopedic medical device to obtain an axis 1;
S1.2, making the implantable orthopedic medical device rotate left and right each at an angle α around the axis 1 to obtain a first fan-shaped region with a central angle of 2α, and equally dividing the first fan-shaped region into 2n fan-shaped regions each with a central angle of α/n;
S1.3, counting a number of points in each of the 2n fan-shaped regions at which a point cloud of the inner curved surface of the implantable orthopedic medical device is located outside the bone, and determining a fan-shaped region with a maximum number of the points as a first target fan-shaped region; and
S1.4, making the implantable orthopedic medical device rotate left and right, such that the implantable orthopedic medical device rotates to the first target fan-shaped region; and S2, making the implantable orthopedic medical device rotate back and forth;
S2.1, making a straight line that crosses the centroid of the implantable orthopedic medical device and is parallel to a second principal axis of the implantable orthopedic medical device to obtain an axis 2;
S2.2, making the implantable orthopedic medical device rotate back and forth each at an angle β around the axis 2 to obtain a second fan-shaped region with a central angle of 2β, and equally dividing the second fan-shaped region into 2m fan-shaped regions each with a central angle of β/m;
S2.3, counting a number of points in each of the 2m fan-shaped regions at which the point cloud of the inner curved surface of the implantable orthopedic medical device is located outside the bone, and determining a fan-shaped region with a maximum number of the points as a second target fan-shaped region; and
S2.4, making the implantable orthopedic medical device rotate back and forth, such that the implantable orthopedic medical device rotates to the second target fan-shaped region.

Further, the method further includes:
calculating a minimum distance from each point on the inner curved surface of the implantable orthopedic medical device to the anatomical adaptation region; and
generating a distance-based heat map based on minimum distances.

Further, the method further includes:
calculating a minimum distance from each point on the inner curved surface of the implantable orthopedic medical device to the anatomical adaptation region; and
calculating an average value of minimum distances, and evaluating adhesion of the bone to the implantable orthopedic medical device with the average value, where a smaller average value indicates a better adhesion.

In a second aspect, the present disclosure provides an apparatus for evaluating anatomical adaptation of an implantable orthopedic medical device, including a processor and a memory storing program codes, wherein the processor performs the stored program codes to:
obtain a first point cloud data of a target bone and a second target point cloud data of an implantable orthopedic medical device;
determine an anatomical adaptation region obtaining module configured to obtain an anatomical adaptation region of a target bone with the implantable orthopedic medical device based on the first point cloud data and the second target point cloud data;
determine a baseline of the anatomical adaptation region and a baseline of the implantable orthopedic medical device for position registration;
conduct the position registration based on the baseline of the anatomical adaptation region and the baseline of the implantable orthopedic medical device;
calculate a minimum distance from each point on an inner curved surface of the implantable orthopedic medical device to the anatomical adaptation region;
calculate an average value of minimum distances, and compare the average value with a predetermined threshold; and
in response to the average value being less than or equal to the predetermined threshold, repair the target bone with the internal vegetation equipment corresponding to the second target point cloud data.

Compared with the prior art, the present disclosure has the following beneficial effects:

If the implantable orthopedic medical device does not fit well with the bone, that is, the adhesion of the to-be-evaluated bone to the implantable orthopedic medical device is not enough, it will cause loosening or detachment of the implantable orthopedic medical device, and even inflammation and infection. Preoperative optimization of adhesion can effectively improve the stability and long-term effectiveness of the implantable orthopedic medical device. The disclosed technical solution objectively evaluates the adhesion of the target bone to the implantable orthopedic medical device device by calculating the average value of the minimum distances between the target bone and the implantable orthopedic medical device and comparing it with a predetermined threshold, without relying on the subjective judgment of the surgeon. In addition, the above technical solutions enable doctors to use suitable implantable orthopedic medical device for bone repair by selecting appropriate implantable according to comparison results. Furthermore, the point cloud data of the implantable orthopedic medical device can be adjusted based on distances and comparison results to manufacture (for example, 3D print) suitable devices to improve adhesion. Through this method, personalized customization of implantable orthopedic medical devices can be achieved.

In the present disclosure, a first point cloud data of a target bone and a second target point cloud data of a implantable orthopedic medical device are obtained; a first determination for determining an anatomical adaptation region of the target bone with an implantable orthopedic medical device is performed based on the first point cloud data and the second target point cloud data, a second determination for determining a baseline of the anatomical adaptation region and a baseline of the implantable orthopedic medical device for position registration are performed, the position registration is conducted based on the baseline of the anatomical adaptation region and the baseline of the implantable orthopedic medical device, a first calculation for calculating a minimum distance from each point on an inner curved surface of the implantable orthopedic medical device to the anatomical adaptation region; a second calculation for calculating an average value of minimum distances is performed, and the average value is compared with a predetermined threshold; in response to the average value being less than or equal to the predetermined threshold, repairing the target bone with the internal vegetation equipment corresponding to the second target point cloud data. The present disclosure can be used to conduct the matching analysis and adhesion evaluation of implantable orthopedic medical devices to a bone before a surgery and guide the selection of an implantable orthopedic medical device in advance, which is of great significance for the treatment of diseases.

### BRIEF DESCRIPTION OF THE DRAWINGS

FIG. 1 is a flow chart of a method for evaluating anatomical adaptation of an implantable orthopedic medical device provided in an embodiment of the present disclosure;

FIG. 2 is a schematic diagram of an anatomical adaptation region;

FIG. 3 is a schematic diagram of directions of three principal axes of a bone (a tibia);

FIGS. 4A-4B area schematic diagram of an implantable orthopedic medical device (a tibial plate) (FIG. 4A) and a point cloud of an inner curved surface of the implantable orthopedic medical device (FIG. 4B);

FIG. 5 is a schematic diagram of a bone (a tibia) and an implantable orthopedic medical device (a tibial plate) after position registration; and

FIG. 6 is a block diagram of an apparatus for evaluating anatomical adaptation of an implantable orthopedic medical device provided in an embodiment of the present disclosure.

### DETAILED DESCRIPTION OF THE EMBODIMENTS

To make the objectives, technical solutions, and advantages of the present disclosure clear, the present disclosure is further described below with reference to the accompanying drawings and specific implementations. Apparently, the described embodiments are merely some rather than all of the embodiments of the present disclosure. All other embodiments obtained by a person of ordinary skill in the art based on the embodiments of the present disclosure without creative efforts shall fall within the protection scope of the present disclosure.

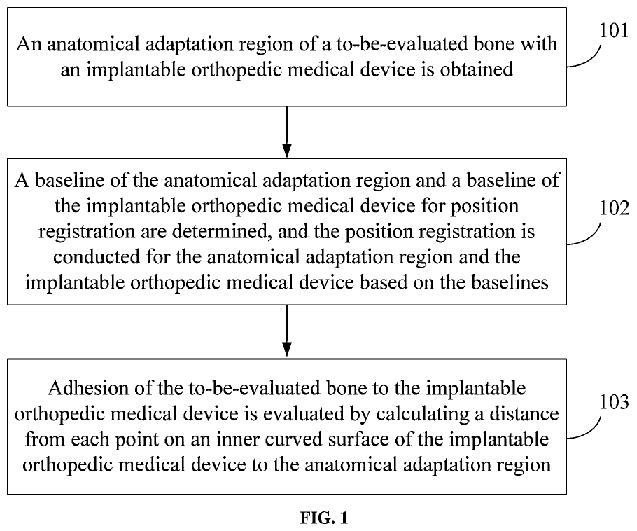

FIG. 1 is a flow chart of a method for evaluating anatomical adaptation of an implantable orthopedic medical device in an embodiment of the present disclosure. The method includes the following steps:
Step 101: An anatomical adaptation region of a to-be-evaluated bone with the implantable orthopedic medical device is obtained.
Step 102: A baseline of the anatomical adaptation region and a baseline of the implantable orthopedic medical device for position registration are determined, and the position registration is conducted for the anatomical adaptation region and the implantable orthopedic medical device based on the baselines.
Step 103: Adhesion of the to-be-evaluated bone to the implantable orthopedic medical device is evaluated by calculating a distance from each point on an inner curved surface of the implantable orthopedic medical device to the anatomical adaptation region.

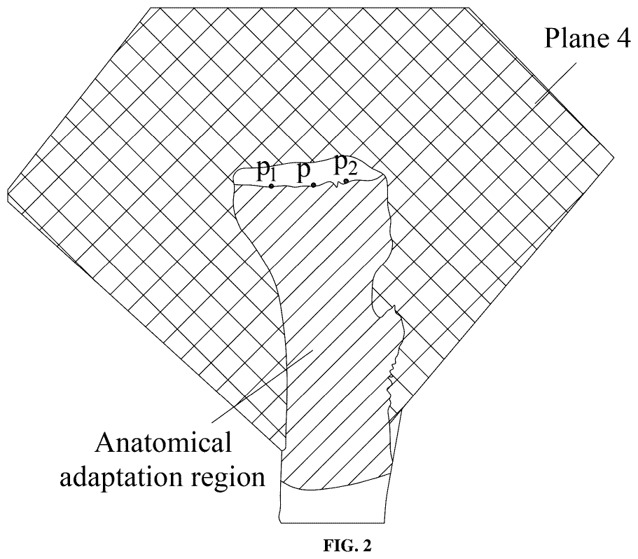

In this embodiment, the step 101 is mainly intended to obtain an anatomical adaptation region of a to-be-evaluated bone with the implantable orthopedic medical device. The anatomical adaptation region refers to a part of a curved surface (plane) of the bone. When the implantable orthopedic medical device is implanted, this part of the curved surface is anatomically adapted to the implantable orthopedic medical device, as shown in FIG. 2. A curved surface of the implantable orthopedic medical device that is adjacent to the anatomical adaptation region (anatomical adaptation) is an inner curved surface of the implantable orthopedic medical device. There are many types of bones to be evaluated and implantable orthopedic medical devices, such as a tibia and a plate. In this embodiment, specific types of the bone and the implantable orthopedic medical device are not restricted.

In this embodiment, the step 102 is mainly intended to conduct position registration. The position registration refers to aligning the implantable orthopedic medical device with the anatomical adaptation region of the bone by adjusting a position of the implantable orthopedic medical device. In this embodiment, the baseline of the anatomical adaptation region and the baseline of the implantable orthopedic medical device for position registration are first determined, and then the two baselines are aligned by adjusting the position of the implantable orthopedic medical device, thereby allowing the position registration. The position registration is conducted to make the bone anatomically adapted well to the implantable orthopedic medical device, such that the adhesion of the bone to the implantable orthopedic medical device can be evaluated accurately.

In this embodiment, the step 103 is mainly intended to evaluate the adhesion of the to-be-evaluated bone to the implantable orthopedic medical device. In this embodiment, after the bone and the implantable orthopedic medical device are subjected to position registration, a distance from each point on the inner curved surface of the implantable orthopedic medical device to the anatomical adaptation region is calculated, and the adhesion of the to-be-evaluated bone to the implantable orthopedic medical device is evaluated based on a size of the distance. The smaller the distance, the better the adhesion.

As an optional embodiment, a process for obtaining the anatomical adaptation region of the to-be-evaluated bone with the implantable orthopedic medical device includes:

Principal component analysis is conducted for point cloud data of the bone to obtain a first principal axis, a second principal axis, and a third principal axis that are perpendicular to each other and correspond to length, width, and height directions of the bone, respectively.

A point p is selected at one end of the bone, and a plane 1 crossing the point p is made with the first principal axis as a normal line.

The plane 1 is translated toward the other end of the bone at a distance to obtain a plane 2.

Points p₁ and p₂ are selected at left and right sides of the point P in the plane 1, respectively, a perpendicular line crossing the point P for a straight line pip₂ is made with a foot point of p₀, and a plane 3 crossing the point p₀ is made with the perpendicular line pp₀ as a normal line.

The plane 3 is translated backward at a distance to obtain a plane 4, and a surface of the bone is intercepted with the planes 1, 2, and 4 to obtain the anatomical adaptation region.

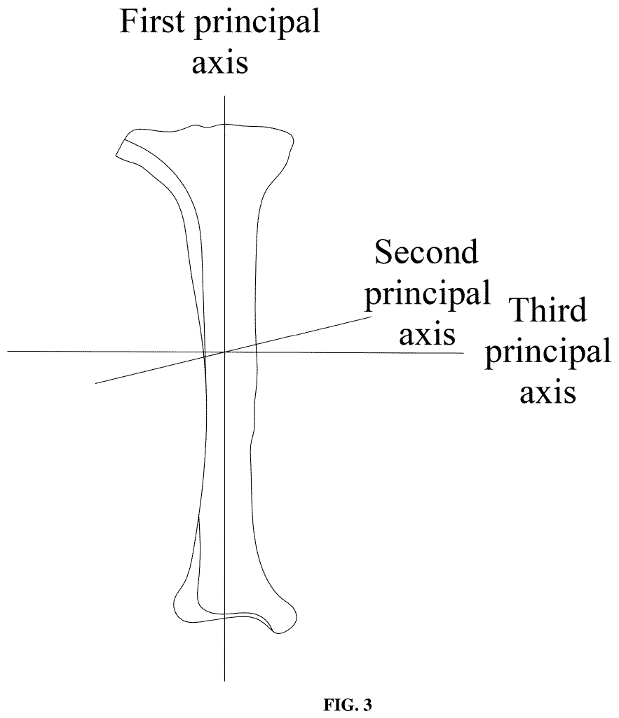

In this embodiment, a technical solution for obtaining an anatomical adaptation region is provided. In this embodiment, a curved surface of the bone is intercepted with the three planes to obtain the anatomical adaptation region. Principal component analysis is first conducted for point cloud data of the bone to obtain three principal axes, namely, a first principal axis, a second principal axis, and a third principal axis that are perpendicular to each other and correspond to length, width, and height directions of the bone, respectively, as shown in FIG. 3. Then, a point p is selected at one end of the bone (which is usually selected by an orthopedic surgeon), and a plane crossing the point p is made with the first principal axis as a normal line to obtain a plane 1. The plane 1 is then translated downward (away from an end of the point p) at a distance to obtain a plane 2. A translated distance is roughly equal to a length of the implantable orthopedic medical device, such as 100 mm. The two upper and lower planes among the three planes have been obtained above. The last plane among the three planes is roughly parallel to the inner curved surface of the implantable orthopedic medical device. A process for obtaining the last plane is as follows: Points p₁ and p₂ located in the plane 1 are selected (which are generally selected by an orthopedic surgeon) respectively at left and right sides of the point p. p₁ and p₂ are connected to obtain a straight line pip₂. A perpendicular line crossing the point P for the straight line pip₂ is made with a foot point of p₀. A plane 3 crossing the point p₀ is made with the perpendicular line pp₀ as a normal line. Then the plane 3 is translated backward at a distance (such as 5 mm) to obtain a plane 4 (as shown in FIG. 2). The plane 4 is the last plane. A curved surface of the bone surrounded by the planes 1, 2, and 4 is the anatomical adaptation region.

As an optional embodiment, a process for determining the baseline of the anatomical adaptation region includes:

A centroid of the bone is calculated.

A straight line that crosses the centroid and is parallel to the first principal axis is made.

A plane 5 that crosses the point p and the straight line is made, and an intersecting line between the plane 5 and the anatomical adaptation region is determined as the baseline of the anatomical adaptation region.

In this embodiment, a technical solution for determining the baseline of the anatomical adaptation region is provided. In this embodiment, the baseline of the anatomical adaptation region can be approximately regarded as a symmetry axis of the anatomical adaptation region (after flattening) along a length direction, and is obtained by intercepting the anatomical adaptation region through a plane. A process for determining the plane is as follows: a centroid of point cloud data of the bone is first calculated, then a straight line that crosses the centroid and is parallel to the first principal axis is made, and a plane crossing the straight line and the point p is the plane (namely, the plane 5).

As an optional embodiment, a process for determining the baseline of the implantable orthopedic medical device includes:

Point cloud data of the inner curved surface of the implantable orthopedic medical device is obtained based on point cloud data of the implantable orthopedic medical device.

Principal component analysis is conducted for the point cloud data of the implantable orthopedic medical device to obtain a first principal axis, a second principal axis, and a third principal axis that are perpendicular to each other and correspond to length, width, and height directions of the implantable orthopedic medical device, respectively.

A centroid of the implantable orthopedic medical device is calculated, and a plane crossing the centroid is made with the second principal axis of the implantable orthopedic medical device as a normal line to obtain a projection plane.

A point cloud of the inner curved surface of the implantable orthopedic medical device is projected to the projection plane to obtain the baseline of the implantable orthopedic medical device.

In this embodiment, a technical solution for determining the baseline of the implantable orthopedic medical device is provided. In this embodiment, the baseline of the implantable orthopedic medical device is obtained by projecting the point cloud of the inner curved surface of the implantable orthopedic medical device to the projection plane. Therefore, the point cloud data of the inner curved surface of the implantable orthopedic medical device is obtained from the point cloud data of the implantable orthopedic medical device (a spatial region), then the projection plane is calculated, and finally the point cloud data of the inner curved surface is projected to the projection plane to obtain the baseline of the implantable orthopedic medical device. A process for calculating the projection plane is as follows: principal component analysis is conducted for the point cloud data of the implantable orthopedic medical device to obtain three principal axes, then a centroid of the implantable orthopedic medical device is calculated, and a plane crossing the centroid is made with a second principal axis as a normal line to obtain the projection plane.

As an optional embodiment, a process for obtaining the point cloud data of the inner curved surface of the implantable orthopedic medical device includes:

Any point on the inner curved surface of the implantable orthopedic medical device is selected, and a normal vector v₀ crossing the point is calculated.

A normal vector v₁ of each point crossing the implantable orthopedic medical device is calculated, where i=1, 2, . . . , N, and N is a number of data points of the implantable orthopedic medical device.

An included angle between v₁ and v₀ is calculated, and the point cloud data of the inner curved surface of the implantable orthopedic medical device is constituted with points corresponding to all included angles smaller than a set threshold.

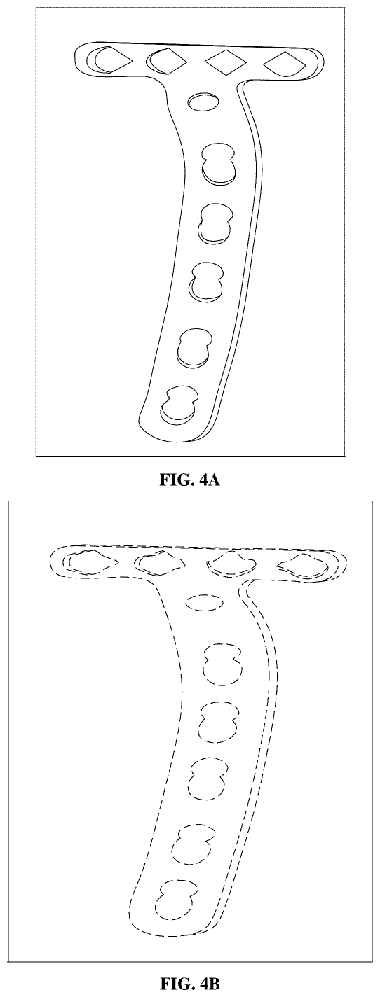

In this embodiment, a technical solution for obtaining the point cloud data of the inner curved surface of the implantable orthopedic medical device is provided. In this embodiment, the point cloud data of the inner curved surface of the implantable orthopedic medical device is obtained from the point cloud data of a spatial region of the implantable orthopedic medical device. A technical principle is as follows: Any point is first selected from the inner curved surface, and then a normal vector v₀ crossing this point is calculated. Then, for the point cloud data of the spatial region of the implantable orthopedic medical device, it is determined one by one whether a point belongs to the inner curved surface. A determination method is as follows: An included angle between the normal vector v₁ and the normal vector v₀ of a point is calculated. If the included angle is smaller than the set threshold, the point belongs to the inner curved surface, otherwise, the point does not belong to the inner curved surface. The set threshold can be 90°. FIGS. 4A-4B are a schematic diagram of point cloud data of a spatial region and a point cloud of an inner curved surface for a tibial plate as an implantable orthopedic medical device.

As an optional embodiment, a process for conducting the position registration for the anatomical adaptation region and the implantable orthopedic medical device based on the baselines includes:

Registration for the baseline of the anatomical adaptation region and the baseline of the implantable orthopedic medical device is conducted by ICP to obtain a transformation matrix R and a translation vector V.

A point cloud matrix of the implantable orthopedic medical device is subjected to the following transformation:

$${A}^{\sim} = A * R + V$$

where A and A⁻ represent point cloud matrices of the implantable orthopedic medical device before and after the transformation, respectively.

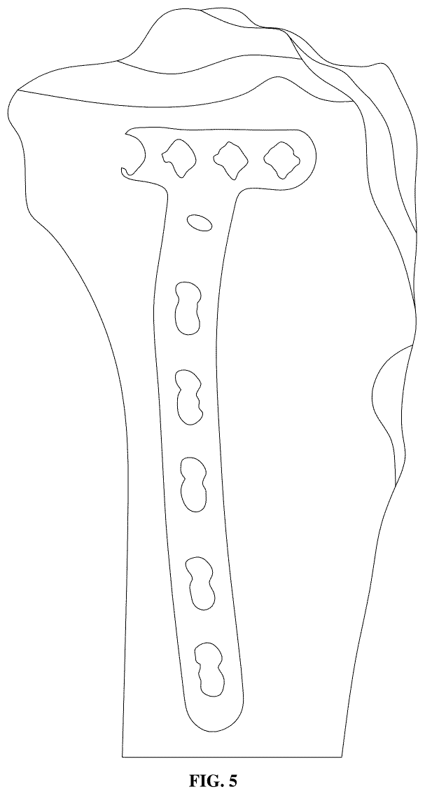

In this embodiment, a technical solution for conducting the position registration is provided. As mentioned above, in this embodiment, the registration is conducted based on the two baselines. In this embodiment, the two baselines are first subjected to ICP to obtain the transformation matrix R and the translation vector V, and then the point cloud data of the implantable orthopedic medical device is subjected to transformation to obtain registered point cloud data of the implantable orthopedic medical device. ICP is a mature prior art, and specific algorithm steps are not given in this embodiment. A schematic diagram of the tibia and the tibial plate after position registration is shown in FIG. 5.

As an optional embodiment, after the position registration is conducted, the method further includes: the implantable orthopedic medical device is subjected to attitude adjustment according to the following steps:
S1: The implantable orthopedic medical device is allowed to rotate left and right:
S1.1: A straight line that crosses a centroid of the implantable orthopedic medical device and is parallel to a first principal axis of the implantable orthopedic medical device is made to obtain an axis 1.
S1.2: The implantable orthopedic medical device is allowed to rotate left and right each at an angle α around the axis 1 to obtain a first fan-shaped region with a central angle of 2α, and the first fan-shaped region is equally divided into 2n fan-shaped regions each with a central angle of α/n.
S1.3: A number of points in each of the 2n fan-shaped regions at which a point cloud of the inner curved surface of the implantable orthopedic medical device is located outside the bone is counted, and a fan-shaped region with a maximum number of the points is determined as a first target fan-shaped region.
S1.4: The implantable orthopedic medical device is allowed to rotate left and right, such that the implantable orthopedic medical device rotates to the first target fan-shaped region.
S2: The implantable orthopedic medical device is allowed to rotate back and forth.
S2.1: A straight line that crosses the centroid of the implantable orthopedic medical device and is parallel to a second principal axis of the implantable orthopedic medical device is made to obtain an axis 2.
S2.2: The implantable orthopedic medical device is allowed to rotate back and forth each at an angle β around the axis 2 to obtain a second fan-shaped region with a central angle of 2β, and the second fan-shaped region is equally divided into 2m fan-shaped regions each with a central angle of β/m.
S2.3: A number of points in each of the 2m fan-shaped regions at which the point cloud of the inner curved surface of the implantable orthopedic medical device is located outside the bone is counted, and a fan-shaped region with a maximum number of the points is determined as a second target fan-shaped region.
S2.4: The implantable orthopedic medical device is allowed to rotate back and forth, such that the implantable orthopedic medical device rotates to the second target fan-shaped region.

In this embodiment, a technical solution for subjecting the implantable orthopedic medical device to attitude adjustment after the position registration is provided. The attitude adjustment of the implantable orthopedic medical device refers to rotations around two axes (axes 1 and 2) parallel to the first and second principal axes at specified angles (a roll angle and a pitch angle), respectively. The attitude adjustment is conducted to further make the bone anatomically adapted well to the implantable orthopedic medical device, such that the adhesion of the bone to the implantable orthopedic medical device can be evaluated accurately. A process for the adjustment at the roll angle around the axis 1 is the same as a process for the adjustment at the pitch angle around the axis 2, and the processes both are as follows: a rotation axis (axes 1 and 2) is first determined, and then a target rotation angle is determined. A process for determining the target rotation angle is a direction of finding a maximum point density where the point cloud of the inner curved surface is located outside the bone. A specific method is as above, which will not be repeated here.

As an optional embodiment, the method further includes:
a minimum distance from each point on the inner curved surface of the implantable orthopedic medical device to the anatomical adaptation region is calculated.

A distance-based heat map is generated based on minimum distances.

In this embodiment, in order to conveniently and intuitively display the adhesion of the bone to the implantable orthopedic medical device, a distance-based heat map is also generated, and a minimum distance from a point on the inner curved surface of the implantable orthopedic medical device to the bone is represented by a color.

An algorithm for generating the distance-based heat map is provided below:

Input: a point P on a point cloud of the inner curved surface of the implantable orthopedic medical device, and a 3D model V of the bone.

Output: The nearest point P′ corresponding to the point P on V.

V is subjected to Octree construction.

A triangle mesh m nearest to P on V is calculated based on P and Octree.

With m as a plane, a perpendicular line crossing the point P for m is made, and an intersecting point between the perpendicular line and m is calculated. The intersecting point is the point P′ nearest to the point P on V.

As an optional embodiment, the method further includes:
a minimum distance from each point on the inner curved surface of the implantable orthopedic medical device to the anatomical adaptation region is calculated.
an average value of minimum distances is calculated, and adhesion of the bone to the implantable orthopedic medical device is evaluated with the average value, where a smaller average value indicates a better adhesion.

In this embodiment, a technical solution for evaluating the adhesion is provided. In this embodiment, a minimum distance from each point on the inner curved surface of the implantable orthopedic medical device to the anatomical adaptation region is calculated, and a calculation method can refer to the previous embodiment. Then an average value of minimum distances is calculated, and adhesion of the bone to the implantable orthopedic medical device is evaluated with the average value. A smaller average value indicates a better adhesion. Of course, the adhesion can also be evaluated with other statistics, such as a mean squared error of the minimum distances. The smaller the mean squared error, the better the adhesion.

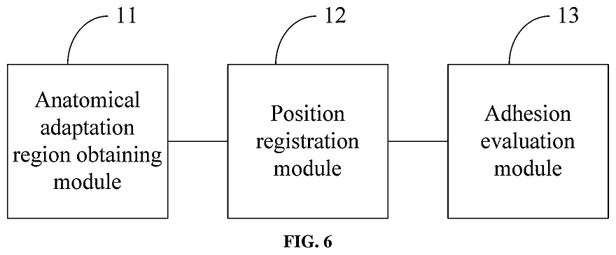

FIG. 6 is a schematic diagram of a constitution of an apparatus for evaluating anatomical adaptation of an implantable orthopedic medical device in an embodiment of the present disclosure. The apparatus includes an anatomical adaptation region obtaining module 11, a position registration module 12, and an adhesion evaluation module 13.

The anatomical adaptation region obtaining module 11 is configured to obtain an anatomical adaptation region of a to-be-evaluated bone with the implantable orthopedic medical device.

The position registration module 12 is configured to determine a baseline of the anatomical adaptation region and a baseline of the implantable orthopedic medical device for position registration and conduct the position registration for the anatomical adaptation region and the implantable orthopedic medical device based on the baselines.

The adhesion evaluation module 13 is configured to evaluate adhesion of the to-be-evaluated bone to the implantable orthopedic medical device by calculating a distance from each point on an inner curved surface of the implantable orthopedic medical device to the anatomical adaptation region.

The apparatus of this embodiment can be configured to execute the technical solution of the method embodiment shown in FIG. 1, and has a similar implementation principle and technical effect to the method embodiment, which are not repeated here.

The above are merely specific implementations of the present disclosure, but the protection scope of the present disclosure is not limited thereto. Any modification or replacement easily conceived by those skilled in the art within the technical scope of the present disclosure should fall within the protection scope of the present disclosure. Therefore, the protection scope of the present disclosure should be subject to the protection scope of the claims.

What is claimed is:

1. A method for evaluating anatomical adaptation of an implantable orthopedic medical device, comprising the following steps:
obtaining a first point cloud data of a target bone and a second target point cloud data of an implantable orthopedic medical device;
performing a first determination for determining an anatomical adaptation region of the target bone with the implantable orthopedic medical device based on the first point cloud data and the second target point cloud data;
performing a second determination for determining a baseline of the anatomical adaptation region and a baseline of the implantable orthopedic medical device for position registration;
conducting the position registration based on the baseline of the anatomical adaptation region and the baseline of the implantable orthopedic medical device;
performing a first calculation for calculating a minimum distance from each point on an inner curved surface of the implantable orthopedic medical device to the anatomical adaptation region;
performing a second calculation for calculating an average value of minimum distances, and comparing the average value with a predetermined threshold;
in response to the average value being less than or equal to the predetermined threshold, repairing the target bone with the internal vegetation equipment corresponding to the second target point cloud data.

2. The method for evaluating anatomical adaptation of an implantable orthopedic medical device according to claim 1, wherein in response to the average value being greater than the predetermined threshold:
generating a distance-based heat map based on the minimum distances and adjusting the second target point cloud data according to the distance-based heat map;
repeating the first determination, the second determination, the position registration, the first calculation, and the second calculation on the adjusted second target cloud data until the adjusted second target cloud data is less than or equal to the predetermined threshold;
repairing the target bone with an internal vegetation equipment manufactured according to the adjusted second target point cloud data.

3. The method for evaluating anatomical adaptation of an implantable orthopedic medical device according to claim 1, wherein a process for determining the anatomical adaptation region of the target bone with the implantable orthopedic medical device comprises:
conducting principal component analysis for point cloud data of the bone to obtain a first principal axis, a second principal axis, and a third principal axis that are perpendicular to each other and correspond to length, width, and height directions of the bone, respectively;
selecting a point p at one end of the bone, and making a plane 1 crossing the point p with the first principal axis as a normal line;
translating the plane 1 toward the other end of the bone at a distance to obtain a plane 2;
selecting points p₁ and p₂ at left and right sides of the point P in the plane 1, respectively, making a perpendicular line crossing the point P for a straight line pip₂ with a foot point of p₀, and making a plane 3 crossing the point p₀ with the perpendicular line pp₀ as a normal line; and
translating the plane 3 backward at a distance to obtain a plane 4, and intercepting a surface of the bone with the planes 1, 2, and 4 to obtain the anatomical adaptation region.

4. The method for evaluating anatomical adaptation of an implantable orthopedic medical device according to claim 3, wherein a process for determining the baseline of the anatomical adaptation region comprises:
calculating a centroid of the bone;
making a straight line that crosses the centroid and is parallel to the first principal axis; and
making a plane 5 that crosses the point p and the straight line, and determining an intersecting line between the plane 5 and the anatomical adaptation region as the baseline of the anatomical adaptation region.

5. The method for evaluating anatomical adaptation of an implantable orthopedic medical device according to claim 3, wherein a process for determining the baseline of the implantable orthopedic medical device comprises:
obtaining point cloud data of the inner curved surface of the implantable orthopedic medical device based on point cloud data of the implantable orthopedic medical device;
conducting principal component analysis for the point cloud data of the implantable orthopedic medical device to obtain a first principal axis, a second principal axis, and a third principal axis that are perpendicular to each other and correspond to length, width, and height directions of the implantable orthopedic medical device, respectively;
calculating a centroid of the implantable orthopedic medical device, and making a plane crossing the centroid with the second principal axis of the implantable orthopedic medical device as a normal line to obtain a projection plane; and
projecting a point cloud of the inner curved surface of the implantable orthopedic medical device to the projection plane to obtain the baseline of the implantable orthopedic medical device.

6. The method for evaluating anatomical adaptation of an implantable orthopedic medical device according to claim 5, wherein a process for obtaining the point cloud data of the inner curved surface of the implantable orthopedic medical device comprises:
selecting any point on the inner curved surface of the implantable orthopedic medical device, and calculating a normal vector v₀ crossing the point;
calculating a normal vector v₁ of each point crossing the implantable orthopedic medical device, wherein i=1, 2, . . . , N, and N is a number of data points of the implantable orthopedic medical device; and
calculating an included angle between v₁ and v₀, and constituting the point cloud data of the inner curved surface of the implantable orthopedic medical device with points corresponding to all included angles smaller than a set threshold.

7. The method for evaluating anatomical adaptation of an implantable orthopedic medical device according to claim 1, wherein a process for conducting the position registration based on the baseline of the anatomical adaptation region and the baseline of the implantable orthopedic medical device comprises:
conducting registration for the baseline of the anatomical adaptation region and the baseline of the implantable orthopedic medical device by iterative closest point (ICP) to obtain a transformation matrix R and a translation vector V; and
subjecting a point cloud matrix of the implantable orthopedic medical device to the following transformation:
$${A}^{\sim} = A * R + V$$
wherein A and A represent point cloud matrices of the implantable orthopedic medical device before and after the transformation, respectively.

8. The method for evaluating anatomical adaptation of an implantable orthopedic medical device according to claim 1, wherein after the position registration is conducted, the method further comprises: subjecting the implantable orthopedic medical device to attitude adjustment according to the following steps:
S1, making the implantable orthopedic medical device rotate left and right;
S1.1, making a straight line that crosses a centroid of the implantable orthopedic medical device and is parallel to a first principal axis of the implantable orthopedic medical device to obtain an axis 1;
S1.2, making the implantable orthopedic medical device rotate left and right each at an angle α around the axis 1 to obtain a first fan-shaped region with a central angle of 2α, and equally dividing the first fan-shaped region into 2n fan-shaped regions each with a central angle of α/n;
S1.3, counting a number of points in each of the 2n fan-shaped regions at which a point cloud of the inner curved surface of the implantable orthopedic medical device is located outside the bone, and determining a fan-shaped region with a maximum number of the points as a first target fan-shaped region; and
S1.4, making the implantable orthopedic medical device rotate left and right, such that the implantable orthopedic medical device rotates to the first target fan-shaped region; and
S2, making the implantable orthopedic medical device rotate back and forth;
S2.1, making a straight line that crosses the centroid of the implantable orthopedic medical device and is parallel to a second principal axis of the implantable orthopedic medical device to obtain an axis 2;
S2.2, making the implantable orthopedic medical device rotate back and forth each at an angle β around the axis 2 to obtain a second fan-shaped region with a central angle of 2β, and equally dividing the second fan-shaped region into 2m fan-shaped regions each with a central angle of β/m;
S2.3, counting a number of points in each of the 2m fan-shaped regions at which the point cloud of the inner curved surface of the implantable orthopedic medical device is located outside the bone, and determining a fan-shaped region with a maximum number of the points as a second target fan-shaped region; and
S2.4, making the implantable orthopedic medical device rotate back and forth, such that the implantable orthopedic medical device rotates to the second target fan-shaped region.

9. The method for evaluating anatomical adaptation of an implantable orthopedic medical device according to claim 1, further comprising:
calculating a minimum distance from each point on the inner curved surface of the implantable orthopedic medical device to the anatomical adaptation region; and
calculating an average value of minimum distances, and evaluating adhesion of the bone to the implantable orthopedic medical device with the average value, wherein a smaller average value indicates a better adhesion.

10. An apparatus for evaluating anatomical adaptation of an implantable orthopedic medical device comprising a processor and a memory storing program codes, wherein the processor performs the stored program codes to:
obtain a first point cloud data of a target bone and a second target point cloud data of an implantable orthopedic medical device;
determine an anatomical adaptation region of a target bone with the implantable orthopedic medical device based on the first point cloud data and the second target point cloud data;
determine a baseline of the anatomical adaptation region and a baseline of the implantable orthopedic medical device for position registration;
conduct the position registration based on the baseline of the anatomical adaptation region and the baseline of the implantable orthopedic medical device;
calculate a minimum distance from each point on an inner curved surface of the implantable orthopedic medical device to the anatomical adaptation region;
calculate an average value of minimum distances, and compare the average value with a predetermined threshold; and
in response to the average value being less than or equal to the predetermined threshold, repair the target bone with the internal vegetation equipment corresponding to the second target point cloud data.
